(19) United States
(12) Patent Application Publication
(10) Pub. No.: US 2025/0325646 A1
(43) Pub. Date: Oct. 23, 2025

(54) COMPOSITIONS AND METHODS FOR INDUCING AN IMMUNE RESPONSE AGAINST EPSTEIN-BARR VIRUS

(71) Applicant: NOVAVAX, INC., Gaithersburg, MD (US)

(72) Inventor: Gregory GLENN, Gaithersburg, MD (US)

(21) Appl. No.: 18/853,030

(22) PCT Filed: Mar. 30, 2023
(86) PCT No.: PCT/US2023/065158

§ 371 Date: Sep. 30, 2024

Related U.S. Application Data
(60) Provisional application No. 63/325,316, filed on Mar. 30, 2022.

(51) Int. Cl. A61K 39/12; A61K 39/00; C07K 14/005; C07K 14/47

(52) U.S. Cl. CPC A61K 39/12; C07K 14/005; C07K 14/47; A61K 2039/55577; A61K 2039/6031; C07K 2319/02; C12N 2710/16222; C12N 2710/16234

(57) ABSTRACT

Provided herein are compositions comprising Epstein-Barr Virus envelope proteins (e.g., glycoprotein 350 (gp350)) and saponin adjuvant and methods of using the aforementioned compositions for inducing an immune response against Epstein-Barr Virus.

### CROSS REFERENCE TO RELATED APPLICATIONS

This application claims priority to U.S. Application No. 63/325,316, filed Mar. 30, 2022. The contents of this application are incorporated by reference in their entirety herein for all purposes.

### DESCRIPTION OF THE TEXT FILE SUBMITTED ELECTRONICALLY

The contents of the electronic sequence listing (NOVV_098_01WO_SeqList_ST26.xml; Size: 55.553 bytes; and Date of Creation: Mar. 30, 2023) are herein incorporated by reference in its entirety.

### FIELD

The present disclosure is generally related to fusion proteins comprising Epstein-Barr Virus (EBV) envelope proteins or a fragment thereof and (ii) ferritin or a fragment thereof and nanoparticles and immunogenic compositions comprising the same, which are useful for stimulating immune responses against EBV. The nanoparticles provide antigens and are typically produced using recombinant approaches. The disclosure also provides compositions containing the nanoparticles, methods for producing them, and methods of stimulating immune responses against EBV.

### BACKGROUND OF THE INVENTION

Infectious diseases remain a problem throughout the world. Epstein-Barr Virus (EBV) causes 125,000 cases of infectious mononucleosis in the United States per year. 13% of patients diagnosed with infectious mononucleosis still have fatigue six months after diagnosis, and 1% have other serious complications. Infectious mononucleosis is a major cause of lost time for military recruits.

EBV is also associated with about 200,000 cancer cases per year and 140,000 cancer deaths worldwide. Specifically, EBV is associated with 84,000 cases of gastric carcinoma, 78,000 cases of nasopharyngeal carcinoma, 29,000 cases of Hodgkin's lymphoma, 7000 cases of Burkitt lymphoma, and 2000 cases of post-transplant lymphoproliferative disease.

Therefore, there is a need in the art for new compositions and methods for preventing EBV infections.

### SUMMARY OF THE INVENTION

The present disclosure provides fusion proteins comprising (i) an Epstein-Barr Virus (EBV) envelope protein or a fragment thereof and (ii) ferritin or a fragment thereof. In embodiments, provided herein are immunogenic compositions comprising the aforementioned fusion proteins for inducing immune responses against EBV. The present disclosure also provides novel methods of administering said immunogenic compositions.

Provided herein is a fusion protein comprising (i) an Epstein-Barr Virus (EBV) envelope protein or a fragment thereof and (ii) ferritin or a fragment thereof. In embodiments, the ferritin or fragment thereof is bulldog ferritin, a Helicobacter pylori ferritin protein, an Escherichia coli ferritin protein, or a combination thereof. In embodiments, the ferritin or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 16-20 and 25-38. In embodiments, the N-terminus of the ferritin protein or fragment thereof comprises a peptide having about 6 to about 14 additional amino acids. In embodiments, the peptide comprises the amino acid sequence of any one of ESQVRQNF (SEQ ID NO: 40), ESQVRQQF (SEQ ID NO: 41), SGESQVRQQF (SEQ ID NO: 42), and SGESQVRQNF (SEQ ID NO: 43). In embodiments, the EBV envelope protein or fragment comprises one or more proteins selected from any one of gp350, gH, gp42, and gL. In embodiments, the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 1-15, 21-22, 39, and 44. In embodiments, the EBV envelope protein or fragment thereof is gp350 or a fragment thereof. In embodiments, the gp350 or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 1-8, 21-22, 39, and 44. In embodiments, the EBV envelope protein or fragment thereof comprises one, two, three, or four mutations selected from any one of W162N, N164T, D208N, and E210T, wherein the amino acids of the EBV envelope protein or fragment thereof are numbered according to SEQ ID NO: 1. In embodiments, the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of SEQ ID NO: 1. In embodiments, the fusion protein comprises: (i) an EBV envelope protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2; and (ii) a ferritin protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38. In embodiments, the fusion protein comprises: (i) an EBV envelope protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 39; and (ii) a ferritin protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38. In embodiments, the fusion protein comprises: (i) an EBV envelope protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 44; and (ii) a ferritin protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38. In embodiments, the fusion protein comprises: (i) an EBV envelope protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 1; and (ii) a ferritin protein or fragment thereof that comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38. In embodiments, the EBV envelope protein or fragment thereof is N-terminal to the ferritin or fragment thereof. In embodiments, the EBV envelope protein or fragment thereof is C-terminal to the ferritin or fragment thereof. In embodiments, the fusion protein comprises a signal peptide. In embodiments, the signal peptide is a bovine prolactin signal peptide or a human CD5 signal peptide.

Provided herein are nucleic acids encoding the fusion proteins described herein. Provided herein are vectors comprising the nucleic acids described herein. Provided herein are host cells expressing the fusion proteins described herein. Provided herein are nanoparticles comprising the fusion proteins described herein. Provided herein are nanoparticles comprising an EBV envelope protein or fragment thereof and a detergent. In embodiments, the detergent is a non-ionic detergent. In embodiments, the non-ionic detergent is polysorbate-20 (PS20), polysorbate-40 (PS40), polysorbate-60 (PS60), polysorbate-65 (PS65), or polysorbate-80 (PS80). In embodiments, the EBV envelope protein or fragment thereof comprises a transmembrane domain. In embodiments, the transmembrane domain has an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 23. In embodiments, the EBV envelope protein or fragment thereof comprises any amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 1 or 44.

Provided herein are immunogenic compositions comprising the fusion proteins described herein. Provided herein are immunogenic compositions comprising the nanoparticles described herein. In embodiments, the immunogenic composition comprises an adjuvant. In embodiments, the adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the immunogenic compositions described herein comprise from about 30 μg to about 60 μg adjuvant. In embodiments, the immunogenic compositions described herein comprise about 49 μg adjuvant. In embodiments, the immunogenic compositions described herein comprise from about 25 μg to about 75 μg fusion protein. In embodiments, the immunogenic compositions described herein comprise about 50 μg fusion protein.

In embodiments, provided herein are methods of stimulating an immune response against Epstein-Barr virus in a subject comprising administering an immunogenic composition described herein. In embodiments, the methods comprise administering multiple doses of the immunogenic composition. In embodiments, the methods comprise administering a first dose, a second dose, and a third dose of the immunogenic composition. In embodiments, the methods comprise administering the second dose of immunogenic composition 30 days after the first dose of immunogenic composition. In embodiments, the methods comprise administering the third dose of immunogenic composition 180 days after the first dose of immunogenic composition. In embodiments, the methods comprise administering the third dose of immunogenic composition 180 days after the second dose of immunogenic composition. In embodiments, the methods comprise administering the immunogenic composition intramuscularly. In embodiments, provided herein are pre-filled syringes comprising immunogenic compositions described herein.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

The term “percent identity” in the context of two or more nucleic acid or polypeptide sequences, refers to two or more sequences or subsequences that have a specified percentage of nucleotides or amino acid residues that are the same, when compared. Percentage identity can be calculated using the tools CLUSTALW2, which are available online. The following parameters may be used for CLUSTALW2 Pairwise alignment: Protein Weight Matrix=Gonnet; Gap Open=10; Gap Extension=0.1.

### Immunogenic Compositions Containing Fusion Proteins Comprising Epstein-Barr Virus Envelope Proteins and Ferritin

### Fusion Proteins

Provided herein are immunogenic compositions comprising fusion proteins comprising (i) an Epstein-Barr Virus (EBV) envelope protein or a fragment thereof and (ii) ferritin or a fragment thereof. In embodiments, the EBV envelope protein or a fragment thereof is located at the N-terminus of the ferritin or fragment thereof. In embodiments, the EBV envelope protein or a fragment thereof is located at the C-terminus of the ferritin or fragment thereof. Alternative fusion proteins that may be utilized in the immunogenic compositions herein are described in the following documents, which are incorporated by reference herein in their entirety for all purposes: U.S. Pat. No. 10,744,199 and Kanekiyo et al. Cell. 2015 Aug. 27; 162 (5): 1090-100.

In embodiments, a “fragment thereof” of a protein is from about 10 to about 1500 amino acids in length (e.g. about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or about 1500 amino acids in length). In embodiments, a fragment thereof comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least 97%, at least about 98%, at least about 99%, or at least about 99.5% of the amino acids of the wild-type version of the protein.

In embodiments, the fusion protein further comprises a protein tag used for purification or detection. In embodiments, the protein tag is a polyglutamate tag, a FLAG-tag, a HA-tag, a polyHis-tag (having about 5-10 histidines) (SEQ ID NO: 45), a hexahistidine tag (SEQ ID NO: 46), an 8X-His-tag (having eight histidines) (SEQ ID NO: 47), a Myc-tag, a Glutathione-S-transferase-tag, a Green fluorescent protein-tag, Maltose binding protein-tag, a Thioredoxin-tag, an Fc-tag, or a C-tag. In embodiments, the extension comprises a C-tag. A C-tag comprises the sequence EPEA (SEQ ID NO: 48).

### Ebv Envelope Proteins

In embodiments, the fusion protein comprises an EBV envelope protein. In embodiments, the EBV envelope protein is selected from the group consisting of gp350, gH, gp42, gL, or combinations thereof.

In embodiments, the EBV envelope protein comprises gp350 or a fragment thereof. In embodiments, the EBV envelope protein comprises the full length gp350 protein. An exemplary full length gp350 protein from Epstein-Barr virus strain B95-8 (UNIPROT ID: P03200) has the amino acid sequence of SEQ ID NO: 1. The full length gp350 protein comprises Domains 1, 2, 3, a receptor binding domain, a transmembrane domain, and a topological domain. In embodiments, the EBV envelope protein comprises one or more fragments of gp350 selected from any one of gp350 Domain 1, EBV gp350 Domain 2, and EBV gp350 Domain 3, the EBV receptor binding domain (RBD), the EBV ectodomain, the EBV transmembrane domain, and the EBV topological domain. In embodiments, the EBV envelope protein lacks the N-terminal methionine.

In embodiments, the EBV envelope protein is the EBV ectodomain. In embodiments, the EBV ectodomain has the sequence of SEQ ID NO: 2.

In embodiments, the EBV envelope protein comprises a transmembrane domain. In embodiments, the transmembrane domain has at least 80%, at least 85%, at least 90%, at least 91%, at least 92%, at least 93%, at least 94%, at least 95%, at least 96%, at least 97%, at least 98%, at least 99%, or 100% identity to a transmembrane domain having the amino acid sequence of SEQ ID NO: 23. In embodiments, the EBV envelope protein comprises a topological domain. In embodiments, the topological domain has at least 80%, at least 85%, at least 90%, at least 91%, at least 92%, at least 93%, at least 94%, at least 95%, at least 96%, at least 97%, at least 98%, at least 99%, or 100% identity to a topological domain having the amino acid sequence of SEQ ID NO: 24. In embodiments, the EBV envelope protein comprises a transmembrane domain and a topological domain.

In embodiments, the fusion protein comprises an EBV envelope protein having at least 80%, at least 85%, at least 90%, at least 91%, at least 92%, at least 93%, at least 94%, at least 95%, at least 96%, at least 97%, at least 98%, at least 99%, or 100% identity to a protein having the amino acid sequence of any one of SEQ ID NOS: 1-15, 21-22, 39, and 44.

In embodiments, an EBV envelope protein or fragment thereof comprises one or more of the following mutations, W162N, N164T, D208N, and E210T, wherein the amino acid sequence of the EBV envelope protein or fragment thereof is numbered according to SEQ ID NO: 1.

In embodiments, an EBV envelope protein or fragment thereof lacks an N-terminal methionine.

Exemplary EBV envelope proteins are provided in Table A.

TABLE A

| SEQ ID | Protein | |
|---|---|---|
| NO: | Name | Amino acid sequence |
| 1 | gp350 | MEAALLVCQYTIQSLIHLTGEDPGFFNVEIPEFPFYPTCNVCTAD |
| | protein | VNVTINFDVGGKKHQLDLDFGQLTPHTKAVYQPRGAFGGSENATN |
| | | LFLLELLGAGELALTMRSKKLPINVTTGEEQQVSLESVDVYFQDV |
| | | FGTMWCHHAEMQNPVYLIPETVPYIKWDNCNSTNITAVVRAQGLD |
| | | VTLPLSLPTSAQDSNFSVKTEMLGNEIDIECIMEDGEISQVLPGD |
| | | NKFNITCSGYESHVPSGGILTSTSPVATPIPGTGYAYSLRLTPRP |
| | | VSRFLGNNSILYVFYSGNGPKASGGDYCIQSNIVFSDEIPASQDM |
| | | PTNTTDITYVGDNATYSVPMVTSEDANSPNVTVTAFWAWPNNTET |
| | | DFKCKWTLTSGTPSGCENISGAFASNRTFDITVSGLGTAPKTLII |
| | | TRTATNATTTTHKVIFSKAPESTTTSPTLNTTGFADPNTTTGLPS |
| | | STHVPTNLTAPASTGPTVSTADVTSPTPAGTTSGASPVTPSPSPW |
| | | DNGTESKAPDMTSSTSPVTTPTPNATSPTPAVTTPTPNATSPTPA |
| | | VTTPTPNATSPTLGKTSPTSAVTTPTPNATSPTLGKTSPTSAVTT |
| | | PTPNATSPTLGKTSPTSAVTTPTPNATGPTVGETSPQANATNHTL |
| | | GGTSPTPVVTSQPKNATSAVTTGQHNITSSSTSSMSLRPSSNPET |
| | | LSPSTSDNSTSHMPLLTSAHPTGGENITQVTPASISTHHVSTSSP |
| | | APRPGTTSQASGPGNSSTSTKPGEVNVTKGTPPQNATSPQAPSGQ |
| | | KTAVPTVTSTGGKANSTTGGKHTTGHGARTSTEPTTDYGGDSTTP |
| | | RPRYNATTYLPPSTSSKLRPRWTFTSPPVTTAQATVPVPPTSQPR |
| | | FSNLSMLVLQWASLAVLTLLLLLVMADCAFRRNLSTSHTYTTPPY |
| | | DDAETYV |
| 2 | gp350 | EAALLVCQYTIQSLIHLTGEDPGFFNVEIPEFPFYPTCNVCTADV |
| | protein | NVTINFDVGGKKHQLDLDFGQLTPHTKAVYQPRGAFGGSENATNL |
| | ectodomain | FLLELLGAGELALTMRSKKLPINVTTGEEQQVSLESVDVYFQDVF |
| | | GTMWCHHAEMQNPVYLIPETVPYIKWDNCNSTNITAVVRAQGLDV |
| | | TLPLSLPTSAQDSNFSVKTEMLGNEIDIECIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVESDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAPESTTTSPTLNTTGFADPNTTTGLPSS |
| | | THVPTNLTAPASTGPTVSTADVTSPTPAGTTSGASPVTPSPSPWD |
| | | NGTESKAPDMTSSTSPVTTPTPNATSPTPAVTTPTPNATSPTPAV |
| | | TTPTPNATSPTLGKTSPTSAVTTPTPNATSPTLGKTSPTSAVTTP |
| | | TPNATSPTLGKTSPTSAVTTPTPNATGPTVGETSPQANATNHTLG |
| | | GTSPTPVVTSQPKNATSAVTTGQHNITSSSTSSMSLRPSSNPETL |
| | | SPSTSDNSTSHMPLLTSAHPTGGENITQVTPASISTHHVSTSSPA |
| | | PRPGTTSQASGPGNSSTSTKPGEVNVTKGTPPQNATSPQAPSGQK |
| | | TAVPTVTSTGGKANSTTGGKHTTGHGARTSTEPTTDYGGDSTTPR |
| | | PRYNATTYLPPSTSSKLRPRWTFTSPPVTTAQATVPVPPTSQPRF |
| | | SNLS |
| 3 | gp350 | EAALLVCQYTIQSLIHLTGEDPGFFNVEIPEFPFYPTCNVCTADV |
| | receptor | NVTINFDVGGKKHQLDLDFGQLTPHTKAVYQPRGAFGGSENATNL |
| | binding | FLLELLGAGELALTMRSKKLPINVTTGEEQQVSLESVDVYFQDVF |
| | domain | GTMWCHHAEMQNPVYLIPETVPYIKWDNCNSTNITAVVRAQGLDV |
| | (RBD) | TLPLSLPTSAQDSNFSVKTEMLGNEIDIECIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVFSDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAPESTTTSPTLNTTGFADPNTTTGLPSS |
| | | THVPTNLTAPASTGPTVST |
| 4 | gp350 | ALLVCQYTIQSLIHLTGEDPGFFNVEIPEFPFYPTCNVCTADVNV |
| | Domain | TINFDVGGKKHQLDLDFGQLTPHTKAVYQPRGAFGGSENATNLFL |
| | 1 | LELLGAGELALTMRSKKLPINVTTGEEQQVSLESVDVYFQDVFGT |
| | | MWCHHAEMQNPVYLI |
| 5 | gp350 | CNSTNITAVVRAQGLDVTLPLSLPTSAQDSNFSVKTEMLGNEIDI |
| | Domain | ECIMEDGEISQVLPGDNKFNITCSGYESHVPSGGILTSTSPVATP |
| | 2 | IPGTGYAYSLRLTPRPVSRFLGNNSILYVFYSGNGPKASGGDYCI |
| | | QSNIVF |
| 6 | gp350 | TNTTDITYVGDNATYSVPMVTSEDANSPNVTVTAFWAWPNNTETD |
| | Domain | FKCKWTLTSGTPSGCENISGAFASNRTFDITVSGLGTAPKTLIIT |
| | 3 | RTATNATTTTHKVIFSKAPE |
| 7 | gp350 | EAALLVCQYTIQSLIHLTGEDPGFFNVEIPEFPFYPTCNVCTADV |
| | Domain | NVTINFDVGGKKHQLDLDFGQLTPHTKAVYQPRGAFGGSENATNL |
| | 1/Domain | FLLELLGAGELALTMRSKKLPINVTTGEEQQVSLESVDVYFQDVF |
| | 2 | GTMWCHHAEMQNPVYLIPETVPYIKWDNCNSTNITAVVRAQGLDV |
| | | TLPLSLPTSAQDSNFSVKTEMLGNEIDIECIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVESDEIPASQDMP |
| | | T |
| 8 | gp350 | EAALLVCQYTIQSLIHLTGEDPGFFNVEIPEFPFYPTCNVCTADV |
| | Domain | NVTINFDVGGKKHQLDLDFGQLTPHTKAVYQPRGAFGGSENATNL |
| | 1/Domain | FLLELLGAGELALTMRSKKLPINVTTGEEQQVSLESVDVYFQDVF |
| | 2/ | GTMWCHHAEMQNPVYLIPETVPYIKWDNCNSTNITAVVRAQGLDV |
| | Domain | TLPLSLPTSAQDSNFSVKTEMLGNEIDIECIMEDGEISQVLPGDN |
| | 3 | KFNITCSGYESHVPSGGILTSTSPVATPIPGTGYAYSLRLTPRPV |
| | | SRFLGNNSILYVFYSGNGPKASGGDYCIQSNIVFSDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAP |
| 9 | gH | MQLLCVFCLVLLWEVGAASLSEVKLHLDIEGHASHYTIPWTELMA |
| | protein | KVPGLSPEALWREANVTEDLASMLNRYKLIYKTSGTLGIALAEPV |
| | | DIPAVSEGSMQVDASKVHPGVISGLNSPACMLSAPLEKQLFYYIG |
| | | TMLPNTRPHSYVFYQLRCHLSYVALSINGDKFQYTGAMTSKFLMG |
| | | TYKRVTEKGDEHVLSLVFGKTKDLPDLRGPFSYPSLTSAQSGDYS |
| | | LVIVTTFVHYANFHNYFVPNLKDMFSRAVTMTAASYARYVLQKLV |
| | | LLEMKGGCREPELDTETLTTMFEVSVAFFKVGHAVGETGNGCVDL |
| | | RWLAKSFFELTVLKDIIGICYGATVKGMQSYGLERLAAMLMATVK |
| | | MEELGHLTTEKQEYALRLATVGYPKAGVYSGLIGGATSVLLSAYN |
| | | RHPLFQPLHTVMRETLFIGSHVVLRELRLNVTTQGPNLALYQLLS |
| | | TALCSALEIGEVLRGLALGTESGLFSPCYLSLRFDLTRDKLLSMA |
| | | PQEATLDQAAVSNAVDGFLGRLSLEREDRDAWHLPAYKCVDRLDK |
| | | VLMIIPLINVTFIISSDREVRGSALYEASTTYLSSSLFLSPVIMN |
| | | KCSQGAVAGEPRQIPKIQNFTRTQKSCIFCGFALLSYDEKEGLET |
| | | TTYITSQEVQNSILSSNYFDFDNLHVHYLLLTTNGTVMEIAGLYE |
| | | ERAHVVLAIILYFIAFALGIFLVHKIVMFFL |
| 10 | gL | MRAVGVFLAICLVTIFVLPTWGNWAYPCCHVTQLRAQHLLALENI |
| | protein | SDIYLVSNQTCDGFSLASLNSPKNGSNQLVISRCANGLNVVSFFI |
| | | SILKRSSSALTGHLRELLTTLETLYGSFSVEDLFGANLNRYAWHR |
| | | GG |
| 11 | gp42 | MVSFKQVRVPLFTAIALVIVLLLAYFLPPRVRGGGRVAAAAITWV |
| | protein | PKPNVEVWPVDPPPPVNFNKTAEQEYGDKEVKLPHWTPTLHTFQV |
| | | PQNYTKANCTYCNTREYTFSYKGCCFYFTKKKHTWNGCFQACAEL |
| | | YPCTYFYGPTPDILPVVTRNLNAIESLWVGVYRVGEGNWTSLDGG |
| | | TFKVYQIFGSHCTYVSKFSTVPVSHHECSFLKPCLCVSQRSNS |
| 12 | Soluble | MPMGSLQPLATLYLLGMLVASVLAGGRVAAAAITWVPKPNVEVWP |
| | gp42 | VDPPPPVNFNKTAEQEYGDKEVKLPHWTPTLHTFQVPQNYTKANC |
| | | TYCNTREYTFSYKGCCFYFTKKKHTWNGCFQACAELYPCTYFYGP |
| | | TPDILPVVTRNLNAIESLWVGVYRVGEGNWTSLDGGTFKVYQIFG |
| | | SHCTYVSKFSTVPVSHHECSFLKPCLCVSQRSNS |
| 13 | gL | MRAVGVFLAICLVTIFVLPTWGNWAYPCCHVTQLRAQHLLALENI |
| | protein | SDIYLVSNQTCDGFSLASLNSPKNGSNQLVISRCANGLNVVSFFI |
| | | SILKRSSSALTGHLRELLTTLETLYGSFSVEDLFGANLNRYAWHR |
| | | GG |
| 14 | Encapsulin- | MPMGSLQPLATLYLLGMLVASMEFLKRSFAPLTEKQWQEIDNRAR |
| | gp42 | EIFKTQLYGRKFVDVEGPYGWEYAAHPLGEVEVLSDENEVVKWGL |
| | | RKSLPLIELRATFTLDLWELDNLERGKPNVDLSSLEETVRKVAEF |
| | | EDEVIFRGCEKSGVKGLLSFEERKIECGSTPKDLLEAIVRALSIF |
| | | SKDGIEGPYTLVINTDRWINFLKEEAGHYPLEKRVEECLRGGKII |
| | | TTPRIEDALVVSERGGDFKLILGQDLSIGYEDREKDAVRLFITET |
| | | FTFQVVNPEALILLKSGSGGGSGGGGGRVAAAAITWVPKPNVEVW |
| | | PVDPPPPVNFNKTAEQEYGDKEVKLPHWTPTLHTFQVPQNYTKAN |
| | | CTYCNTREYTFSYKGCCFYFTKKKHTWNGCFQACAELYPCTYFYG |
| | | PTPDILPVVTRNLNAIESLWVGVYRVGEGNWTSLDGGTFKVYQIF |
| | | GSHCTYVSKFSTVPVSHHECSFLKPCLCVSQRSNS |
| 15 | Soluble | MQLLCVFCLVLLWEVGAASLSEVKLHLDIEGHASHYTIPWTELMA |
| | gH | KVPGLSPEALWREANVTEDLASMLNRYKLIYKTSGTLGIALAEPV |
| | | DIPAVSEGSMQVDASKVHPGVISGLNSPACMLSAPLEKQLFYYIG |
| | | TMLPNTRPHSYVFYQLRCHLSYVALSINGDKFQYTGAMTSKFLMG |
| | | TYKRVTEKGDEHVLSLVFGKTKDLPDLRGPFSYPSLTSAQSGDYS |
| | | LVIVTTFVHYANFHNYFVPNLKDMFSRAVTMTAASYARYVLQKLV |
| | | LLEMKGGCREPELDTETLTTMFEVSVAFFKVGHAVGETGNGCVDL |
| | | RWLAKSFFELTVLKDIIGICYGATVKGMQSYGLERLAAMLMATVK |
| | | MEELGHLTTEKQEYALRLATVGYPKAGVYSGLIGGATSVLLSAYN |
| | | RHPLFQPLHTVMRETLFIGSHVVLRELRLNVTTQGPNLALYQLLS |
| | | TALCSALEIGEVLRGLALGTESGLFSPCYLSLRFDLTRDKLLSMA |
| | | PQEATLDQAAVSNAVDGFLGRLSLEREDRDAWHLPAYKCVDRLDK |
| | | VLMIIPLINVTFIISSDREVRGSALYEASTTYLSSSLFLSPVIMN |
| | | KCSQGAVAGEPRQIPKIQNFTRTQKSCIFCGFALLSYDEKEGLET |
| | | TTYITSQEVQNSILSSNYFDFDNLHVHYLLLTTNGTVMEIAGLYE |
| | | ERAH |
| 21 | | EAALLVCQYTIQSLIHLTGEDPGFFNVEIPEFPFYPTCNVCTADV |
| | | NVTINFDVGGKKHQLDLDFGQLTPHTKAVYQPRGAFGGSENATNL |
| | | FLLELLGAGELALTMRSKKLPINVTTGEEQQVSLESVDVYFQDVF |
| | | GTMWCHHAEMQNPVYLIPETVPYIKWDNCNSTNITAVVRAQGLDV |
| | | TLPLSLPTSAQDSNFSVKTEMLGNEIDIECIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVFSDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAPESTTTSPTLNTTGFADPNTTTGLPSS |
| | | THVPTNLTAPASTGPTVSTADVTSPTPAGTTSGASPVTPSPSPWD |
| | | NGTESKAPDMTSSTSPVTTPTPNATSPTPAVTTPTPNATSPTPAV |
| | | TTPTPNATSPTLGKTSPTSAVTTPTPNATSPTLGKTSPTSAVTTP |
| | | TPNATSPTLGKTSPTSAVTTPTPNATGPTVGETSPQANATNHTLG |
| | | GTSPTPVVTSQPKNATSAVTTGQHNITSSSTSSMSLRPSSNPETL |
| | | SPSTSDNSTSHMPLLTSAHPTGGENITQVTPASISTHHVSTSSPA |
| | | PRPGTTSQASGPGNSSTSTKPGEVNVTKGTPPQNATSPQAPSGQK |
| | | TAVPTVTSTGGKANSTTGGKHTTGHGARTSTEPTTDYGGDSTTPR |
| | | PRYNATTYLPPSTSSKLRPRWTFTSPPVTTAQATVPVPPTSQPRF |
| | | SNLSMLVLQWASLAVLTLLLLLVMADCAFRRNLSTSHTYTTPPYD |
| | | DAETYV |
| 22 | | MEAALLVCQYTIQSLIHLTGEDPGFFNVEIPEFPFYPTCNVCTAD |
| | | VNVTINFDVGGKKHQLDLDFGQLTPHTKAVYQPRGAFGGSENATN |
| | | LFLLELLGAGELALTMRSKKLPINVTTGEEQQVSLESVDVYFQDV |
| | | FGTMWCHHAEMQNPVYLIPETVPYIKWDNCNSTNITAVVRAQGLD |
| | | VTLPLSLPTSAQDSNFSVKTEMLGNEIDIECIMEDGEISQVLPGD |
| | | NKFNITCSGYESHVPSGGILTSTSPVATPIPGTGYAYSLRLTPRP |
| | | VSRFLGNNSILYVFYSGNGPKASGGDYCIQSNIVFSDEIPASQDM |
| | | PTNTTDITYVGDNATYSVPMVTSEDANSPNVTVTAFWAWPNNTET |
| | | DFKCKWTLTSGTPSGCENISGAFASNRTFDITVSGLGTAPKTLII |
| | | TRTATNATTTTHKVIFSKAPESTTTSPTLNTTGFADPNTTTGLPS |
| | | STHVPTNLTAPASTGPTVSTADVTSPTPAGTTSGASPVTPSPSPW |
| | | DNGTESKAPDMTSSTSPVTTPTPNATSPTPAVTTPTPNATSPTPA |
| | | VTTPTPNATSPTLGKTSPTSAVTTPTPNATSPTLGKTSPTSAVTT |
| | | PTPNATSPTLGKTSPTSAVTTPTPNATGPTVGETSPQANATNHTL |
| | | GGTSPTPVVTSQPKNATSAVTTGQHNITSSSTSSMSLRPSSNPET |
| | | LSPSTSDNSTSHMPLLTSAHPTGGENITQVTPASISTHHVSTSSP |
| | | APRPGTTSQASGPGNSSTSTKPGEVNVTKGTPPQNATSPQAPSGQ |
| | | KTAVPTVTSTGGKANSTTGGKHTTGHGARTSTEPTTDYGGDSTTP |
| | | RPRYNATTYLPPSTSSKLRPRWTFTSPPVTTAQATVPVPPTSQPR |
| | | FSNLS |
| 39 | Non- | EAALLVCQYTIQSLIHLTGEDPGFFNVEIPEFPFYPTCNVCTADV |
| | naturally | NVTINFDVGGKKHQLDLDFGQLTPHTKAVYQPRGAFGGSENATNL |
| | occurring | FLLELLGAGELALTMRSKKLPINVTTGEEQQVSLESVDVYFQDVF |
| | gp350 | GTMWCHHAEMQNPVYLIPETVPYIKNDTCNSTNITAVVRAQGLDV |
| | protein | TLPLSLPTSAQDSNFSVKTEMLGNEINITCIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVFSDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAPESTTTSPTLNTTGFADPNTTTGLPSS |
| | | THVPTNLTAPASTGPTVSTADVTSPTPAGTTSGASPVTPSPSPWD |
| | | NGTESKAPDMTSSTSPVTTPTPNATSPTPAVTTPTPNATSPTPAV |
| | | TTPTPNATSPTLGKTSPTSAVTTPTPNATSPTLGKTSPTSAVTTP |
| | | TPNATSPTLGKTSPTSAVTTPTPNATGPTVGETSPQANATNHTLG |
| | | GTSPTPVVTSQPKNATSAVTTGQHNITSSSTSSMSLRPSSNPETL |
| | | SPSTSDNSTSHMPLLTSAHPTGGENITQVTPASISTHHVSTSSPA |
| | | PRPGTTSQASGPGNSSTSTKPGEVNVTKGTPPQNATSPQAPSGQK |
| | | TAVPTVTSTGGKANSTTGGKHTTGHGARTSTEPTTDYGGDSTTPR |
| | | PRYNATTYLPPSTSSKLRPRWTFTSPPVTTAQATVPVPPTSQPRF |
| | | SNLS |
| 44 | gp350 | EAALLVCQYTIQSLIHLTGEDPGFFNVEIPEFPFYPTCNVCTADV |
| | protein | NVTINFDVGGKKHQLDLDFGQLTPHTKAVYQPRGAFGGSENATNL |
| | | FLLELLGAGELALTMRSKKLPINVTTGEEQQVSLESVDVYFQDVF |
| | | GTMWCHHAEMQNPVYLIPETVPYIKWDNCNSTNITAVVRAQGLDV |
| | | TLPLSLPTSAQDSNFSVKTEMLGNEIDIECIMEDGEISQVLPGDN |
| | | KFNITCSGYESHVPSGGILTSTSPVATPIPGTGYAYSLRLTPRPV |
| | | SRFLGNNSILYVFYSGNGPKASGGDYCIQSNIVFSDEIPASQDMP |
| | | TNTTDITYVGDNATYSVPMVTSEDANSPNVTVTAFWAWPNNTETD |
| | | FKCKWTLTSGTPSGCENISGAFASNRTFDITVSGLGTAPKTLIIT |
| | | RTATNATTTTHKVIFSKAPESTTTSPTLNTTGFADPNTTTGLPSS |
| | | THVPTNLTAPASTGPTVSTADVTSPTPAGTTSGASPVTPSPSPWD |
| | | NGTESKAPDMTSSTSPVTTPTPNATSPTPAVTTPTPNATSPTPAV |
| | | TTPTPNATSPTLGKTSPTSAVTTPTPNATSPTLGKTSPTSAVTTP |
| | | TPNATSPTLGKTSPTSAVTTPTPNATGPTVGETSPQANATNHTLG |
| | | GTSPTPVVTSQPKNATSAVTTGQHNITSSSTSSMSLRPSSNPETL |
| | | SPSTSDNSTSHMPLLTSAHPTGGENITQVTPASISTHHVSTSSPA |
| | | PRPGTTSQASGPGNSSTSTKPGEVNVTKGTPPQNATSPQAPSGQK |
| | | TAVPTVTSTGGKANSTTGGKHTTGHGARTSTEPTTDYGGDSTTPR |
| | | PRYNATTYLPPSTSSKLRPRWTFTSPPVTTAQATVPVPPTSQPRF |
| | | SNLSMLVLQWASLAVLTLLLLLVMADCAFRRNLSTSHTYTTPPYD |
| | | DAETYV |

### Ferritin

In embodiments, the fusion protein comprises ferritin or a fragment thereof. In embodiments, the ferritin or fragment thereof is bulldog ferritin, a Helicobacter pylori ferritin protein, an Escherichia coli ferritin protein, or a combination thereof. In embodiments, the fusion protein comprises a ferritin protein having at least 80%, at least 85%, at least 90%, at least 91%, at least 92%, at least 93%, at least 94%, at least 95%, at least 96%, at least 97%, at least 98%, at least 99%, or 100% identity to a protein having the amino acid sequence of any one of SEQ ID NOS: 16-20 and 25-38. In embodiments, the ferritin protein comprises from 6 to 14 additional amino acids at the N-terminus. In embodiments, the N-terminus of the ferritin protein comprises any one of ESQVRQNF (SEQ ID NO: 40), ESQVRQQF (SEQ ID NO: 41), SGESQVRQQF (SEQ ID NO: 42), and SGESQVRQNF (SEQ ID NO: 43).

Exemplary ferritin proteins are provided in Table B. In embodiments, the ferritin protein has a UniProt ID of P07797 or Q9ZLI1.

TABLE B

| SEQ | | |
|---|---|---|
| ID | | |
| NO: | Protein Name | Amino acid sequence |
| 16 | Helicobacter | MLSKDIIKLLNEQVNKEMNSSNLYMSMSSWCYTHSLDGAG |
| | pylori ferritin | LFLFDHAAEEYEHAKKLIIFLNENNVPVQLTSISAPEHKF |
| | protein | EGLTQIFQKAYEHEQHISESINNIVDHAIKSKDHATFNFL |
| | | QWYVAEQHEEEVLFKDILDKVELIGNENHGLYLADQYVKG |
| | | IAKSRKS |
| 17 | Escherichia | MLKPEMIEKLNEQMNLELYSSLLYQQMSAWCSYHTFEGAA |
| | coli ferritin | AFLRRHAQEEMTHMQRLFDYLTDTGNLPRINTVESPFAEY |
| | protein | SSLDELFQETYKHEQLITQKINELAHAAMTNQDYPTFNFL |
| | | QWYVSEQHEEEKLFKSIIDKLSLAGKSGEGLYFIDKELST |
| | | LDAQN |
| 18 | Bulldog | MESQVRQNFHQDCEAGLNRTVNLKFHSSYVYLSMASYFNR |
| | ferritin | DDVALSNFAKFFRERSEEEKEHAEKLIEYQNQRGGRVFLQ |
| | | SVEKPERDDWANGLEALQTALKLQKSVNQALLDLHAVAAD |
| | | KSDPHMTDFLESPYLSESVETIKKLGDHITSLKKLWSSHP |
| | | GMAEYLFNKHTLG |
| 19 | Helicobacter | SGESQVRQQFSKDIEKLLNEQVNKEMQSSNLYMSMSSWCY |
| | pylori ferritin | THSLDGAGLFLFDHAAEEYEHAKKLIIFLNENNVPVQLTS |
| | protein/Bulldog | ISAPEHKFEGLTQIFQKAYEHEQHISESINNIVDHAIKSK |
| | ferritin | DHATFNFLQWYVAEQHEEEVLFKDILDKIELIGNENHGLY |
| | fusion protein | LADQYVKGIAKSRKSGS |
| 20 | Escherichia | SGESQVRQNFKPEMEEKLNEQMNLELYSSLLYQQMSAWCS |
| | coli ferritin | YHTFEGAAAFLRRHAQEEMTHMQRLFDYLTDTGNLPRINT |
| | protein/Bulldog | VESPFAEYSSLDELFQETYKHEQLITQKINELAHAAMTNQ |
| | ferritin | DYPTFNFLQWYVSEQHEEEKLFKSIIDKLSLAGKSGEGLY |
| | fusion protein | FIDKELSTLDGS |
| 25 | Helicobacter | MLSKDIIKLLNEQVNKEMNSSNLYMSMSSWCYTHSLDGAG |
| | pylori non- | LFLFDHAAEEYEHAKKLIIFLNENNVPVQLTSISAPEHKF |
| | heme ferritin | EGLTQIFQKAYEHEQHISESINNIVDHAIKSKDHATFNFL |
| | protein | QWYVAEQHEEEVLFKDILDKIELIGNENHGLYLADQYVKG |
| | | IAKSRKS |
| 26 | Helicobacter | SKDIIKLLNEQVNKEMNSSNLYMSMSSWCYTHSLDGAGLF |
| | pylori non- | LFDHAAEEYEHAKKLIIFLNENNVPVQLTSISAPEHKFEG |
| | heme ferritin | LTQIFQKAYEHEQHISESINNIVDHAIKSKDHATFNFLQW |
| | protein | YVAEQHEEEVLFKDILDKIELIGNENHGLYLADQYVKGIA |
| | | KSRKS |
| 27 | Non-naturally | MLSKDIEKLLNEQVNKEMNSSNLYMSMSSWCYTHSLDGAG |
| | occurring | LFLFDHAAEEYEHAKKLIIFLNENNVPVQLTSISAPEHKF |
| | ferritin protein | EGLTQIFQKAYEHEQHISESINNIVDHAIKSKDHATFNFL |
| | | QWYVAEQHEEEVLFKDILDKIELIGNENHGLYLADQYVKG |
| | | IAKSRKS |
| 28 | Non-naturally | SKDIEKLLNEQVNKEMNSSNLYMSMSSWCYTHSLDGAGLF |
| | occurring | LFDHAAEEYEHAKKLIIFLNENNVPVQLTSISAPEHKFEG |
| | ferritin protein | LTQIFQKAYEHEQHISESINNIVDHAIKSKDHATFNFLQW |
| | | YVAEQHEEEVLFKDILDKIELIGNENHGLYLADQYVKGIA |
| | | KSRKS |
| 29 | Escherichia | KPEMIEKLNEQMNLELYSSLLYQQMSAWCSYHTFEGAAAF |
| | coli ferritin | LRRHAQEEMTHMQRLFDYLTDTGNLPRINTVESPFAEYSS |
| | protein | LDELFQETYKHEQLITQKINELAHAAMTNQDYPTFNFLQW |
| | | YVSEQHEEEKLFKSIIDKLSLAGKSGEGLYFIDKELSTLD |
| | | AQN |
| 30 | Non-naturally | KPEMEEKLNEQMNLELYSSLLYQQMSAWCSYHTFEGAAAF |
| | occurring | LRRHAQEEMTHMQRLFDYLTDTGNLPRINTVESPFAEYSS |
| | ferritin protein | LDELFQETYKHEQLITQKINELAHAAMTNQDYPTFNFLQW |
| | | YVSEQHEEEKLFKSIIDKLSLAGKSGEGLYFIDKELSTLD |
| | | GS |
| 31 | Helicobacter | SKDIIKLLNEQVNKEMNSSNLYMSMSSWCYTHSLDGAGLF |
| | pylori ferritin | LFDHAAEEYEHAKKLIIFLNENNVPVQLTSISAPEHKFEG |
| | protein | LTQIFQKAYEHEQHISESINNIVDHAIKSKDHATFNFLQW |
| | | YVAEQHEEEVLFKDILDKVELIGNENHGLYLADQYVKGIA |
| | | KSRKS |
| 32 | Non-naturally | MLKPEMEEKLNEQMNLELYSSLLYQQMSAWCSYHTFEGAA |
| | occurring | AFLRRHAQEEMTHMQRLFDYLTDTGNLPRINTVESPFAEY |
| | ferritin protein | SSLDELFQETYKHEQLITQKINELAHAAMTNQDYPTFNFL |
| | | QWYVSEQHEEEKLFKSIIDKLSLAGKSGEGLYFIDKELST |
| | | LDGS |
| 33 | Bulldog | SQVRQNFHQDCEAGLNRTVNLKFHSSYVYLSMASYFNRDD |
| | ferritin | VALSNFAKFFRERSEEEKEHAEKLIEYQNQRGGRVFLQSV |
| | | EKPERDDWANGLEALQTALKLQKSVNQALLDLHAVAADKS |
| | | DPHMTDFLESPYLSESVETIKKLGDHITSLKKLWSSHPGM |
| | | AEYLFNKHTLG |
| 34 | Non-naturally | SKDIEKLLNEQVNKEMQSSNLYMSMSSWCYTHSLDGAGLF |
| | occurring | LFDHAAEEYEHAKKLIIFLNENNVPVQLTSISAPEHKFEG |
| | ferritin protein | LTQIFQKAYEHEQHISESINNIVDHAIKSKDHATFNFLQW |
| | | YVAEQHEEEVLFKDILDKIELIGNENHGLYLADQYVKGIA |
| | | KSRKSGS |
| 35 | Non-naturally | MLSKDIEKLLNEQVNKEMQSSNLYMSMSSWCYTHSLDGAG |
| | occurring | LFLFDHAAEEYEHAKKLIIFLNENNVPVQLTSISAPEHKF |
| | ferritin protein | EGLTQIFQKAYEHEQHISESINNIVDHAIKSKDHATFNFL |
| | | QWYVAEQHEEEVLFKDILDKIELIGNENHGLYLADQYVKG |
| | | IAKSRKSGS |
| 36 | Non-naturally | MLSKDIEKLLNEQVNKEMQSSNLYMSMSSWCYTHSLDGAG |
| | occurring | LFLFDHAAEEYEHAKKLIIFLNENNVPVQLTSISAPEHKF |
| | ferritin protein | EGLTQIFQKAYEHEQHISESINNIVDHAIKSKDHATFNFL |
| | | QWYVAEQHEEEVLFKDILDKIELIGNENHGLYLADQYVKG |
| | | IAKSRKS |
| 37 | Non-naturally | SKDIEKLLNEQVNKEMQSSNLYMSMSSWCYTHSLDGAGLF |
| | occurring | LFDHAAEEYEHAKKLIIFLNENNVPVQLTSISAPEHKFEG |
| | ferritin protein | LTQIFQKAYEHEQHISESINNIVDHAIKSKDHATFNFLQW |
| | | YVAEQHEEEVLFKDILDKIELIGNENHGLYLADQYVKGIA |
| | | KSRKS |
| 38 | Non-naturally | ESQVRQQFSKDIEKLLNEQVNKEMQSSNLYMSMSSWCYTH |
| | occurring | SLDGAGLFLFDHAAEEYEHAKKLIIFLNENNVPVQLTSIS |
| | ferritin protein | APEHKFEGLTQIFQKAYEHEQHISESINNIVDHAIKSKDH |
| | | ATFNFLQWYVAEQHEEEVLFKDILDKIELIGNENHGLYLA |
| | | DQYVKGIAKSRKS |

### Nanoparticles Containing Fusion Proteins Described Herein Nanoparticles Containing Ferritin

In embodiments, the fusion proteins form nanoparticles. In embodiments, nanoparticles are produced according to the protocol outlined in the following publication, which is incorporated by reference herein in its entirety for all purpose: Kanekiyo et al. Cell. 2015 Aug. 27; 162 (5): 1090-100. In embodiments, host cells (e.g., 293F or Expi293F cells) are transfected with expression vectors encoding the fusion proteins described herein. In embodiments, nanoparticles are purified by affinity chromatography. In embodiments, a Galanthus nivalis agglutinin resin is used for purification. In embodiments, size exclusion chromatography is used for purification.

### Nanoparticles Containing Detergents

In embodiments, the fusion proteins form nanoparticles comprising the fusion protein and a detergent. In embodiments, provided herein are nanoparticles comprising an EBV envelope protein or fragment thereof and a detergent. In embodiments, the detergent is a non-ionic detergent. In embodiments, the detergent is selected from the group consisting of polysorbate-20 (PS20), polysorbate-40 (PS40), polysorbate-60 (PS60), polysorbate-65 (PS65) and polysorbate-80 (PS80). In embodiments, the EBV envelope protein comprises an ectodomain and a transmembrane domain. In embodiments the transmembrane domain of the EBV envelope protein or fragment thereof interacts with the detergent core, whereas the ectodomain of the EBV envelope protein or fragment thereof projects outward. In embodiments, the nanoparticles are formed using a detergent exchange approach, wherein a first detergent is used to isolate a protein and then that first detergent is exchanged for a second detergent to form the nanoparticles.

The fusion proteins or EBV envelope proteins or fragments thereof contained in the nanoparticles are typically produced by recombinant expression in host cells. Standard recombinant techniques may be used. In embodiments, the fusion proteins or EBV envelope proteins or fragments thereof are expressed in insect host cells using a baculovirus system. In embodiments, the baculovirus is a cathepsin-L knock-out baculovirus, a chitinase knock-out baculovirus. Optionally, the baculovirus is a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusiani cells, e.g. High Five cells, and Drosophila S2 cells. In embodiments, the fusion proteins or EBV envelope proteins or fragments thereof described herein are produced in any suitable host cell. In embodiments, the host cell is an insect cell. In embodiments, the insect cell is an Sf9 cell.

Typical transfection and cell growth methods can be used to culture the cells. Vectors, e.g., vectors comprising polynucleotides that encode fusion proteins or EBV envelope proteins or fragments thereof, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

After growth of the host cells, the fusion proteins or EBV envelope proteins or fragments thereof may be harvested from the host cells using detergents and purification protocols. Once the host cells have grown for 48 to 96 hours, the cells are isolated from the media and a detergent-containing solution is added to solubilize the cell membrane, releasing the protein in a detergent extract. Triton X-100 and TERGITOL® nonylphenol ethoxylate, also known as NP-9, are each preferred detergents for extraction. The detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different first detergents may be used to isolate the fusion proteins or EBV envelope proteins or fragments thereof from the host cell. For example, the first detergent may be Bis(polyethylene glycol bis [imidazoylcarbonyl]), nonoxynol-9, Bis(polyethylene glycol bis [imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CREMOPHOR® EL Macrogolglycerol ricinoleate, Decaethyleneglycol monododecyl ether, N-Decanoyl-N-methylglucamine, n-Decyl alpha-Dglucopyranoside, Decyl beta-D-maltopyranoside, n-Dodecanoyl-N-methylglucamide, nDodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-0-(N-heptylcarbamoyl)-alpha-D-glucopyranoside, Nonaethylene glycol monododecyl ether, N-Nonanoyl-N-methylglucamine, N-NonanoyIN-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGITOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON™ X-100 Polyethylene glycol tert-octylphenyl ether or combinations thereof.

The nanoparticles may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). Preferably, the methods disclosed herein do not use a detergent extraction column; for example a hydrophobic interaction column. Such a column is often used to remove detergents during purification but may negatively impact the methods disclosed here.

To form nanoparticles, the first detergent, used to extract the fusion proteins or EBV envelope proteins or fragments thereof from the host cell is substantially replaced with a second detergent to arrive at the nanoparticle structure. NP-9 is a preferred extraction detergent. Typically, the nanoparticles do not contain detectable NP-9 when measured by HPLC. The second detergent is typically selected from the group consisting of PS20, PS40, PS60, PS65, and PS80. Preferably, the second detergent is PS80.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins—a large family of homologous proteins,” FASEB J. 1990 November; 4 (14): 3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin, is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the fusion protein nanoparticle or EBV envelope protein or fragment thereof form trimers. In embodiments, the fusion protein nanoparticle or EBV envelope protein or fragment thereof trimers are anchored in detergent. In embodiments, each fusion protein nanoparticle or EBV envelope protein or fragment thereof nanoparticle contains at least one trimer associated with a non-ionic core.

The detergent, when incubated with the protein to form the nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the nanoparticle contains about 0.03% to about 0.05% PS80. In embodiments, the nanoparticle contains about 0.01% (v/v) PS80.

In embodiments, purified fusion proteins or EBV envelope proteins or fragments thereof are dialyzed. In embodiments, dialysis occurs after purification. In embodiments, the purified fusion proteins or EBV envelope proteins or fragments thereof are dialyzed in a solution comprising sodium phosphate, NaCl, and PS80. In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 500 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM NaCl. In embodiments, the dialysis solution comprising PS80 comprises about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the dialysis solution comprises about 25 mM sodium phosphate (pH 7.2), about 300 mM NaCl, and about 0.01% (v/v) PS80.

Detergent exchange may be performed with proteins purified as discussed above and purified, frozen for storage, and then thawed for detergent exchange. Stability of compositions disclosed herein may be measured in a variety of ways. In one approach, a peptide map may be prepared to determine the integrity of the antigen protein after various treatments designed to stress the nanoparticles by mimicking harsh storage conditions. Thus, a measure of stability is the relative abundance of antigen peptides in a stressed sample compared to a control sample. For example, the stability of nanoparticles containing the purified fusion proteins or EBV envelope proteins or fragments thereof may be evaluated by exposing the nanoparticles to various pHs, proteases, salt, oxidizing agents, including but not limited to hydrogen peroxide, various temperatures, freeze/thaw cycles, and agitation. It is thought that the position of the glycoprotein anchored into the detergent core provides enhanced stability by reducing undesirable interactions. For example, the improved protection against protease-based degradation may be achieved through a shielding effect whereby anchoring the glycoproteins into the core at the molar ratios disclosed herein results in steric hindrance blocking protease access. Stability may also be measured by monitoring intact proteins.

### Adjuvants

In embodiments, the immunogenic compositions comprise an adjuvant.

In certain embodiments, the compositions disclosed herein may be combined with one or more adjuvants to enhance an immune response. In other embodiments, the compositions are prepared without adjuvants, and are thus available to be administered as adjuvant-free compositions.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

In embodiments, the adjuvant is a saponin adjuvant.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

The saponin fraction may be integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In embodiments, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

The ISCOM matrix or ISCOM complex may comprise from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In some aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-M™ the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

Other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### Excipients

The immunogenic compositions described herein may comprise various excipients, buffers, and the like. For example, the immunogenic compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate is present. Histidine may be present about 0.1% (w/v), about 0.5% (w/v), about 0.7% (w/v), about 1% (w/v), about 1.5% (w/v), about 2% (w/v), or about 2.5% (w/v). Sodium chloride, when present, may be about 150 mM. In certain compositions, the sodium chloride may be present in higher concentrations, for example from about 200 mM to about 500 mM. In embodiments, the sodium chloride is present in a high concentration, including but not limited to about 200 mM, about 250 mM, about 300 mM, about 350 mM, about 400 mM, about 450 mM, or about 500 mM.

### Administration and Dosage

Provided herein are methods for inducing an immune response against EBV in a subject in need thereof comprising administering an immunogenic composition comprising a fusion protein described herein. In embodiments, the subject is human. In embodiments, the subject has a body mass index (BMI) that is less than or equal to 36. In embodiments, the BMI is less than about 35, less than about 34, less than about 33, less than about 32, less than about 31, less than about 30, less than about 29, less than about 28, less than about 27; less than about 26; less than about 25; less than about 24; less than about 23; less than about 22; less than about 21; less than about 20; less than about 19; less than about 18; less than about 17; less than about 16; less than about 15; less than about 14; less than about 13; less than about 12, less than about 11, or less than about 10. In embodiments, the BMI is from 10 to 36, from 15 to 36, from 18.5 to 36, from 25.0 to 29.9, from 18.5 to 24.9, or 30 or higher. In embodiments, the subject is greater than about 60, greater than about 61, greater than about 62, greater than about 63, greater than about 64, greater than about 65, greater than about 66, greater than about 67, greater than about 68, greater than about 69, or greater than about 70 years old. In embodiments, the subject is about 18 to about 29 years old. In embodiments, the subject is aged about 30 to about 65 years old. In embodiments, the subject is about 10 to about 17 years old. In embodiments, the subject is about 1 years old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, about 17 years old, about 18 years old, about 19 years old, about 20 years old, about 21 years old, about 22 years old, about 23 years old, about 24 years old, about 25 years old, about 26 years old, about 27 years old, about 28 years old, about 29 years old, about 30 years old, about 31 years old, about 32 years old, about 33 years old, about 34 years old, about 35 years old, about 36 years old, about 37 years old, about 38 years old, about 39 years old, about 40 years old, about 41 years old, about 42 years old, about 43 years old, about 44 years old, about 45 years old, about 46 years old, about 47 years old, about 48 years old, about 49 years old, about 50 years old, about 51 years old, about 52 years old, about 53 years old, about 54 years old, about 55 years old, about 56 years old, about 57 years old, about 58 years old, about 59 years old, about 60 years old, about 61 years old, about 62 years old, about 63 years old, about 64 years old, about 65 years old, about 66 years old, about 67 years old, about 68 years old, about 69 years old, about 70 years old, about 71 years old, about 72 years old, about 73 years old, about 74 years old, about 75 years old, about 76 years old, about 77 years old, about 78 years old, about 79 years old, about 80 years old, about 81 years old, about 82 years old, about 83 years old, about 84 years old, about 85 years old, about 86 years old, about 87 years old, about 88 years old, about 89 years old, about 90 years old, about 91 years old, about 92 years old, about 93 years old, about 94 years old, about 95 years old, about 96 years old, about 97 years old, about 98 years old, about 99 years old, about 100 years old, including all ages and ranges therebetween.

In embodiments, the each dose of the fusion protein independently comprises from about 0.1 μg to about 100 μg of fusion protein, including all ranges and subranges therebetween. In embodiments, the method comprises administering a fusion protein described herein at a dose of 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg. In embodiments, the fusion protein comprises an EBV envelope protein having an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 1-15, 21-22, 39, and 44. In embodiments, the fusion protein comprises a ferritin protein having an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 16-20 and 25-38.

In embodiments, the immunogenic compositions comprise an adjuvant. In embodiments, the dose of adjuvant ranges from about 1 μg to about 100 μg. For example, in embodiments, the dose of adjuvant is about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg. In embodiments, the adjuvant is a saponin adjuvant. In embodiments, the saponin adjuvant comprises 85% w/w Fraction A ISCOM matrix and 15% w/w Fraction C ISCOM matrix. In embodiments, the adjuvant is a saponin adjuvant. In embodiments, the saponin adjuvant comprises 92% w/w Fraction A ISCOM matrix and 8% w/w Fraction C ISCOM matrix.

In embodiments, the immunogenic compositions described herein are administered in a single dose. In embodiments, the immunogenic compositions described herein are administered in multiple doses. For example, in embodiments, one, two, three, four, five, six, seven, eight, nine, or ten doses of the immunogenic composition is administered. These doses are referred to as a first, second, third, fourth, fifth, sixth, seventh, eight, ninth, or tenth dose.

In embodiments, the second, third, fourth, fifth, sixth, seventh, eight, ninth, or tenth dose of an immunogenic composition is administered about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks after administration of the first dose. In embodiments, the second, third, fourth, fifth, sixth, seventh, eight, ninth, or tenth dose of an immunogenic composition is administered about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 114 days, about 115 days, about 116 days, about 117 days, about 118 days, about 119 days, about 120 days, about 121 days, about 122 days, about 123 days, about 124 days, about 125 days, about 126 days, about 127 days, about 128 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, about 201 days, about 202 days, about 203 days, about 204 days, about 205 days, about 206 days, about 207 days, about 208 days, about 209 days, about 210 days, about 211 days, about 212 days, about 213 days, about 214 days, about 215 days, about 216 days, about 217 days, about 218 days, about 219 days, about 220 days, about 221 days, about 222 days, about 223 days, about 224 days, about 225 days, about 226 days, about 227 days, about 228 days, about 229 days, about 230 days, about 231 days, about 232 days, about 233 days, about 234 days, about 235 days, about 236 days, about 237 days, about 238 days, about 239 days, about 240 days, about 241 days, about 242 days, about 243 days, about 244 days, about 245 days, about 246 days, about 247 days, about 248 days, about 249 days, about 250 days, about 251 days, about 252 days, about 253 days, about 254 days, about 255 days, about 256 days, about 257 days, about 258 days, about 259 days, about 260 days, about 261 days, about 262 days, about 263 days, about 264 days, about 265 days, about 266 days, about 267 days, about 268 days, about 269 days, about 270 days, about 271 days, about 272 days, about 273 days, about 274 days, about 275 days, about 276 days, about 277 days, about 278 days, about 279 days, about 280 days, about 281 days, about 282 days, about 283 days, about 284 days, about 285 days, about 286 days, about 287 days, about 288 days, about 289 days, about 290 days, about 291 days, about 292 days, about 293 days, about 294 days, about 295 days, about 296 days, about 297 days, about 298 days, about 299 days, about 300 days, about 301 days, about 302 days, about 303 days, about 304 days, about 305 days, about 306 days, about 307 days, about 308 days, about 309 days, about 310 days, about 311 days, about 312 days, about 313 days, about 314 days, about 315 days, about 316 days, about 317 days, about 318 days, about 319 days, about 320 days, about 321 days, about 322 days, about 323 days, about 324 days, about 325 days, about 326 days, about 327 days, about 328 days, about 329 days, about 330 days, about 331 days, about 332 days, about 333 days, about 334 days, about 335 days, about 336 days, about 337 days, about 338 days, about 339 days, about 340 days, about 341 days, about 342 days, about 343 days, about 344 days, about 345 days, about 346 days, about 347 days, about 348 days, about 349 days, about 350 days, about 351 days, about 352 days, about 353 days, about 354 days, about 355 days, about 356 days, about 357 days, about 358 days, about 359 days, about 360 days, about 361 days, about 362 days, about 363 days, about 364 days, or about 365 days after administration of the first dose.

In embodiments, three doses of an immunogenic composition described herein are administered, a first dose, a second dose, and a third dose. In embodiments, the second dose is administered 30 days after administration of the first dose, and the third dose is administered 180 days after administration of the first dose. In embodiments, the immunogenic composition comprises about 50 μg of a fusion protein described herein and about 49 μg adjuvant. In embodiments, the adjuvant is a saponin adjuvant.

Compositions disclosed herein may be administered via a systemic route or a mucosal route or a transdermal route or directly into a specific tissue. As used herein, the term “systemic administration” includes parenteral routes of administration. In particular, parenteral administration includes intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

In embodiments, a dose is administered in a volume of about 0.1 mL to about 1.5 mL, for example, about 0.1 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL.

In embodiments, an immunogenic composition may comprise a fusion protein concentration of about 1 μg/mL to about 50 μg/mL, 10 μg/mL to about 100 μg/mL, about 10 μg/mL to about 50 μg/mL, about 175 μg/mL to about 325 μg/mL, about 200 μg/mL to about 300 μg/mL, about 220 μg/mL to about 280 μg/mL, or about 240 μg/mL to about 260 μg/mL.

In some embodiments, the disclosure provides co-formulation (i.e., pre-filled syringes or pre-mix) strategies for immunogenic compositions comprising a fusion protein described herein and an adjuvant (e.g., a saponin adjuvant). Typical vaccine administration strategies currently being utilized are bedside mix formulations. That is, vaccine compositions and adjuvants are stored separately and are mixed prior to administration. Pre-mix, co-formulation, or pre-filled syringe strategies for vaccine are less common due to the concerns of the stability of the antigens (e.g., a fusion protein described herein) and their subsequent immunogenic capabilities. The present disclosure provides immunogenic compositions that can be pre-mixed and stored in advance. The disclosed vaccination strategies and formulations may improve the efficiency of vaccination and may reduce the risks of bedside mixing errors, while maintaining the overall safety and immunogenicity.

A variety of containers may be used to store and transport the pre-mix formulations, including syringes for single administrations and plastic ampules. In some instances, plastic ampules can be manufactured using the blow-fill-seal manufacturing technique or method. In general, the blow-fill-seal (BFS) manufacturing method includes extruding a plastic material (e.g., resin) to form a parison, which is then placed into a mold and cut to size. A filling needle or mandrel is then used to inflate the plastic, which in turn, results in a hollow ampule that substantially conforms to the shape of the mold. Once inflated, a desired volume of liquid can be injected into the ampule, the filling needle or mandrel can be removed, and the ampule can be sealed. Accordingly, BFS can be an automated process that can be performed in a sterile environment without direct human intervention.

In some instances, the ability to aseptically manufacture sterile ampules containing a desired liquid can make BFS manufactured ampules particularly well suited for the pharmaceutical industry. BFS technology, however, has not been compatible with all pharmaceutical liquids, products, etc. For example, some known BFS manufacturing methods include delivering the liquid or product into the ampule while the plastic is still relatively hot, which can result in adverse effects to temperature sensitive liquids and/or products such as vaccines, biologics, etc. Advances in cool BFS technology, however, have increased the variety of suitable products, liquids, etc. allowing some vaccines, biologics, and/or other temperature sensitive pharmaceuticals to be contained in BFS ampules.

In some instances, a BFS ampule can have a size, shape, and/or configuration that is at least partially based on a desired use and/or a desired pharmaceutical liquid or dosage that the ampule is configured to contain. For example, some known BFS ampules can include a pierce through top, a twist-off top, a top including a male or female luer, and/or the like. Some known BFS ampules can have a size and/or shape based on volume of the liquid or dosage configured to be disposed therein. In addition, some known BFS ampules can be manufactured in a strip of multiple, temporarily connected ampules, which can increase manufacturing, packaging, and/or storing efficiencies and/or the like.

In embodiments, the immunogenic compositions described herein are provided in pre-filled syringes. When the immunogenic composition is prepared in a pre-filled syringe, the fusion proteins described herein and adjuvant are combined in advance of administration. In embodiments, the pre-filled syringe contains a fusion protein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, the pre-filled syringe contains a fusion protein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the pre-filled syringe contains a fusion protein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina accounts for at least about 75% by weight and fraction C of Quillaja Saponaria Molina accounts for the remainder of the sum of weights of fraction A of Quillaja Saponaria Molina and fraction C of Quillaja Saponaria Molina in the adjuvant.

### EXAMPLES

### Example 1: A Phase I, Open-Label Study to Evaluate the Efficacy of an Immunogenic Composition Comprising a Fusion Protein Comprising Gp350 and Ferritin Protein Adjuvanted with Saponin Adjuvant

Purpose: The efficacy of immunogenic compositions comprising a fusion protein and a saponin adjuvant are being evaluated in adults aged 18-29 years old. The fusion protein comprises a gp350 protein or fragment thereof and a ferritin protein or a fragment thereof.

Methods: The immunogenic composition is administered intramuscularly at days 0, 30, and 180. Each dose of immunogenic composition comprises 50 μg of fusion protein and 49 μg of saponin adjuvant. The saponin adjuvant comprises 85% w/w Fraction A ISCOM matrix and 15% w/w Fraction C ISCOM matrix. The ability of the compositions to induce an immune response against EBV is evaluated.

Inclusion Criteria: Patients were included in the study if they met the following criteria: (1) aged 18 to 29 years old; (2) the patient agreed not to receive a live vaccine 30 days before or after each dose of immunogenic composition; (3) the patient agreed not to receive an inactivated, subunit, a replication defective vaccine, or a COVID-19 vaccine 14 days before or after administration of the immunogenic composition; (4) hemoglobin and white blood cell count within institutional limits; (5) total lymphoycte count greater than or equal to 800 cells/mm³; (6) platelet count from 125,000 to 500,000/Mm³; (7) alanine aminotransferase less than or equal to 1.25 times the upper limit of normal; (8) serum IgG greater than 600 mg/dL; and a negative human immunodeficiency virus (HIV) test.

Exclusion Criteria: (1) women who are breastfeeding or planning to become pregnant while participating through 60 days after the last dose of vaccine; (2) participant who has received any of the following: (a) More than 10 days of systemic immunosuppressive medications (>=10 mg prednisone dose or its equivalent) or cytotoxic medication within the 30 days prior to first dose of vaccine or immunomodulating therapy within 180 days prior to first dose of vaccine; (b) Blood products, including immunoglobulin products, within 120 days prior to first dose of immunogenic composition; (c) Any live attenuated vaccination within 30 days prior to first dose of immunogenic composition; (d) Medically indicated subunit inactivated or replication-defective vaccines, e.g. influenza, pneumococcal, COVID-19 within 14 days of the first dose of immunogenic composition; (e) Investigational research agents within 30 days prior to first dose or planning to receive investigational products while on study; (f) Allergy treatment with antigen injections, unless on a maintenance schedule of shots no more frequently than once per month; (3) participant that has any of: (a) Febrile illness within 14 days of the first dose of vaccine; (b) Body Mass Index (BMI) >36; (c) Serious reactions to vaccines; (d) Hereditary, acquired, or idiopathic forms of angioedema; (e) Idiopathic urticaria within the past year; (f) Asthma that is not well-controlled or required emergent care, urgent care, hospitalization or intubation during the past two years or that requires the use of oral or intravenous steroids; (g) Diabetes mellitus type 1 or type 2, excluding a history of gestational diabetes; (h) Clinically significant autoimmune disease or immunodeficiency; (i) Hypertension that is not well-controlled; (j) Thyroid disease that is not well-controlled; (k) Bleeding disorder diagnosed by doctor (e.g. factor deficiency, coagulopathy, or platelet disorder requiring special precautions); (l) Significant bruising or bleeding difficulties with IM injections or blood draws; (m) Malignancy that is active or treated malignancy for which there is no reasonable assurance of sustained cure or malignancy that is likely to recur during the study period; (n) Seizure disorder other than a history of 1) febrile seizures 2) seizures secondary to alcohol withdrawal more than 3 years ago, or 3) seizures that have not required treatment within the past 3 years; (o) Asplenia, functional asplenia or any condition resulting in absence or removal of the spleen; (p) History of Guillain-Barre Syndrome; and (q) Alcohol or drug abuse or addiction.

### NUMBERED EMBODIMENTS

1. A fusion protein comprising (i) an Epstein-Barr Virus (EBV) envelope protein or a fragment thereof and (ii) ferritin or a fragment thereof.

2. The fusion protein of embodiment 1, wherein the ferritin or fragment thereof is bulldog ferritin, a Helicobacter pylori ferritin protein, an Escherichia coli ferritin protein, or a combination thereof.

3. The fusion protein of embodiment 1 or 2, wherein the EBV envelope protein is selected from the group consisting of gp350, gH, gp42, and gL.

4. The fusion protein of any one of embodiments 1-3, wherein the EBV envelope protein comprises a gp350 or a fragment thereof comprising one or more domains selected from EBV gp350 Domain 1, EBV gp350 Domain 2, and EBV gp350 Domain 3, the EBV receptor binding domain (RBD), the EBV ectodomain, and combinations thereof.

5. The fusion protein of any one of embodiments 1-4, wherein the EBV envelope protein comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 1-15.

6. The fusion protein of any one of embodiments 1-5, wherein ferritin or a fragment thereof comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 16-20.

7. A nucleic acid encoding a fusion protein of any one of embodiments 1-6.

8. A host cell expressing a fusion protein of any one of embodiments 1-6.

9. An immunogenic composition comprising a fusion protein of any one of embodiments 1-6.

10. The immunogenic composition of embodiment 9, comprising an adjuvant.

11. The immunogenic composition of embodiment 10, wherein the adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

12. The immunogenic composition of embodiment 11, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.

13. The immunogenic composition of embodiment 11, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.

14. The immunogenic composition of any one of embodiments 9-12, wherein the adjuvant is administered at a dose of from about 30 μg to about 60 μg.

15. The immunogenic composition of any one of embodiments 9-12, wherein the adjuvant is administered at a dose of about 49 μg.

16. The immunogenic composition of any one of embodiments 9-12, wherein the EBV envelope protein is administered at a dose of from about 25 μg to about 75 μg.

17. The immunogenic composition of any one of embodiments 9-12, wherein the EBV envelope protein is administered at a dose of about 50 μg.

18. A pre-filled syringe comprising the immunogenic composition of any one of embodiments 9-17.

19. A method of stimulating an immune response against Epstein-Barr virus in a subject comprising administering the immunogenic composition of any one of embodiments 9-17.

20. The method of embodiment 19, comprising administering an adjuvant.

21. The method of embodiment 20, wherein the adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

22. The method of embodiment 21, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.

23. The method of embodiment 21, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.

24. The method of any one of embodiments 20-23, wherein the adjuvant is administered at a dose of from about 30 μg to about 60 μg.

25. The method of any one of embodiments 20-24, wherein the adjuvant is administered at a dose of about 49 μg.

26. The method of any one of embodiments 19-25, wherein the EBV envelope protein is administered at a dose of from about 25 μg to about 75 μg.

27. The method of any one of embodiments 19-25, wherein the EBV envelope protein is administered at a dose of about 50 μg.

28. The method of any one of embodiments 19-27, wherein multiple doses of immunogenic composition are administered.

29. The method of any one of embodiments 19-27, wherein a single dose of or immunogenic composition is administered.

30. The method of embodiment 28, wherein three doses of immunogenic composition are administered.

31. The method of embodiment 30, wherein the first dose is administered on day 0, the second dose is administered on day 30, and the third dose is measured on day 180.

32. The method of any one of embodiments 19-31, wherein the immunogenic composition is administered intramuscularly.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. This application hereby incorporates by reference the disclosure of U.S. Pat. No. 10,744,199 in its entirety for all purposes.

1-47. (canceled)

48. A fusion protein comprising (i) an Epstein-Barr Virus (EBV) envelope protein or a fragment thereof with an amino acid sequence having at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 1-15, 21-22, 39, and 44 and (ii) ferritin or a fragment thereof with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 16-20 and 25-38.

49. The fusion protein of claim 48, wherein the ferritin protein or fragment thereof includes an N-terminus comprising a peptide having an amino acid sequence of any one of ESQVRQNF (SEQ ID NO: 40), ESQVRQQF (SEQ ID NO: 41), SGESQVRQQF (SEQ ID NO: 42), and SGESQVRQNF (SEQ ID NO: 43).

50. The fusion protein of claim 48, wherein the EBV envelope protein or fragment thereof is gp350 or a fragment thereof with an amino acid sequence having at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of any one of SEQ ID NOS: 1-8, 21-22, 39, and 44.

51. The fusion protein of claim 48, wherein the EBV envelope protein or fragment thereof comprises one, two, three, or four mutations selected from any one of W162N, N164T, D208N, and E210T, wherein the amino acids of the EBV envelope protein or fragment thereof are numbered according to SEQ ID NO: 1.

52. The fusion protein of claim 48, wherein the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to a polypeptide having an amino acid sequence of SEQ ID NO: 1.

53. The fusion protein of a claim 48, wherein:
(i) the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2; and
(ii) the ferritin protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38.

54. The fusion protein of claim 48, wherein:
(i) the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 39; and
(ii) the ferritin protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38.

55. The fusion protein of claim 48, wherein:
(i) the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 44; and
(ii) the ferritin protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38.

56. The fusion protein of claim 48, wherein:
(i) the EBV envelope protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 1; and
(ii) the ferritin protein or fragment thereof comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 38.

57. The fusion protein of claim 48, comprising a signal peptide which is a bovine prolactin signal peptide or a human CD5 signal peptide.

58. A nanoparticle comprising a fusion protein of claim 48, comprising an EBV envelope protein or fragment thereof and a non-ionic detergent.

59. The nanoparticle of claim 58, wherein the non-ionic detergent is polysorbate-20 (PS20), polysorbate-40 (PS40), polysorbate-60 (PS60), polysorbate-65 (PS65), or polysorbate-80 (PS80).

60. The nanoparticle of claim 58, wherein the transmembrane domain has an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 23.

61. The nanoparticle of 58, wherein the EBV envelope protein or fragment thereof comprises any amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOS: 1 or 44.

62. An immunogenic composition comprising a fusion protein of claim 48 and a pharmaceutically acceptable buffer.

63. An immunogenic composition comprising the nanoparticle of claim 58 and a pharmaceutically acceptable buffer.

64. The immunogenic composition of claim 62, further comprising an adjuvant with at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

65. The immunogenic composition of claim 64, comprising from about 30 μg to about 60 μg adjuvant.

66. A method of stimulating an immune response against Epstein-Barr virus in a subject comprising administering the immunogenic composition of claim 48.

67. A pre-filled syringe comprising an immunogenic composition of claim 48.


SEQUENCE LISTING (48 sequences)

SEQ ID NO: 1 - protein, 907 aa, Human gammaherpesvirus 4
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 2 - protein, 859 aa, Human gammaherpesvirus 4
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 3 - protein, 469 aa, Human gammaherpesvirus 4
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVST

SEQ ID NO: 4 - protein, 150 aa, Human gammaherpesvirus 4
ALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLI

SEQ ID NO: 5 - protein, 141 aa, Human gammaherpesvirus 4
CNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVF

SEQ ID NO: 6 - protein, 110 aa, Human gammaherpesvirus 4
TNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPE

SEQ ID NO: 7 - protein, 316 aa, Human gammaherpesvirus 4
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPT

SEQ ID NO: 8 - protein, 424 aa, Human gammaherpesvirus 4
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAP

SEQ ID NO: 9 - protein, 706 aa, Human gammaherpesvirus 4
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 10 - protein, 137 aa, Human gammaherpesvirus 4
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 11 - protein, 223 aa, Human gammaherpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 12 - protein, 214 aa, synthetic construct
MPMGSLQPLATLYLLGMLVASVLAGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 13 - protein, 137 aa, Human gammaherpesvirus 4; identical to SEQ ID NO: 10

SEQ ID NO: 14 - protein, 485 aa, synthetic construct
MPMGSLQPLATLYLLGMLVASMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGSGGGSGGGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 15 - protein, 679 aa, synthetic construct
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAH

SEQ ID NO: 16 - protein, 167 aa, Helicobacter pylori
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 17 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 18 - protein, 173 aa, Lithobates catesbeianus
MESQVRQNFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 19 - protein, 177 aa, synthetic construct
SGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 20 - protein, 172 aa, synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 21 - protein, 906 aa, Human gammaherpesvirus 4
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 22 - protein, 860 aa, Human gammaherpesvirus 4
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 23 - protein, 47 aa, Human gammaherpesvirus 4
MLVLQWASLAVLTLLLLLVMADCAFRRNLSTSHTYTTPPYDDAETYV

SEQ ID NO: 24 - protein, 26 aa, Human gammaherpesvirus 4
DCAFRRNLSTSHTYTTPPYDDAETYV

SEQ ID NO: 25 - protein, 167 aa, Helicobacter pylori
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 26 - protein, 165 aa, Helicobacter pylori
SKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 27 - protein, 167 aa, synthetic construct
MLSKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 28 - protein, 165 aa, synthetic construct
SKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 29 - protein, 163 aa, Escherichia coli
KPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 30 - protein, 162 aa, synthetic construct
KPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 31 - protein, 165 aa, Helicobacter pylori
SKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 32 - protein, 164 aa, synthetic construct
MLKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 33 - protein, 171 aa, Lithobates catesbeianus
SQVRQNFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 34 - protein, 167 aa, synthetic construct
SKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 35 - protein, 169 aa, synthetic construct
MLSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 36 - protein, 167 aa, synthetic construct
MLSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 37 - protein, 165 aa, synthetic construct
SKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 38 - protein, 173 aa, synthetic construct
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 39 - protein, 859 aa, synthetic construct
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKNDTCNSTNITAVVRAQGLDVTLPLSLPTSAQDSNFSVKTEMLGNEINITCIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 40 - protein, 8 aa, synthetic construct
ESQVRQNF

SEQ ID NO: 41 - protein, 8 aa, synthetic construct
ESQVRQQF

SEQ ID NO: 42 - protein, 10 aa, synthetic construct
SGESQVRQQF

SEQ ID NO: 43 - protein, 10 aa, synthetic construct
SGESQVRQNF

SEQ ID NO: 44 - protein, 906 aa, Human gammaherpesvirus 4; identical to SEQ ID NO: 21

SEQ ID NO: 45 - protein, 10 aa, synthetic construct
  VARIANT 6..10: note = individual amino acids may be present or absent
HHHHHHHHHH

SEQ ID NO: 46 - protein, 6 aa, synthetic construct
HHHHHH

SEQ ID NO: 47 - protein, 8 aa, synthetic construct
HHHHHHHH

SEQ ID NO: 48 - protein, 4 aa, synthetic construct
EPEA